(19) United States
(12) Patent Application Publication
(10) Pub. No.: US 2014/0171433 A1
(43) Pub. Date: Jun. 19, 2014

(54) MULTIPLE MYELOMA TREATMENT

(71) Applicants: Christopher John Burns, Caulfield North (AU); Andrew Spencen, South Yarra (AU); Katherine Anne Monaghan, Melbourne (AU)

(72) Inventors: Christopher John Burns, Caulfield North (AU); Andrew Spencen, South Yarra (AU); Katherine Anne Monaghan, Melbourne (AU)

(73) Assignee: YM Biosciences Australia Pty Ltd, Foster City (CA)

(21) Appl. No.: 14/115,084

(22) PCT Filed: May 1, 2012
(86) PCT No.: PCT/AU2012/000462

§ 371 Date: Mar. 6, 2014

Related U.S. Application Data
(60) Provisional application No. 61/481,425, filed on May 2, 2011.

(51) Int. Cl. C07D 239/42

(52) U.S. Cl. CPC C07D 239/42
USPC 514/235.8; 544/122

(57) ABSTRACT

A method of treating a subject presenting with multiple myeloma at a stage characterized by an increase in the prevalence of MM cells that (1) are IL-6 non-responsive and/or (2) have a CD45− phenotype, comprising administering to the subject an amount of a compound of formula Ib.

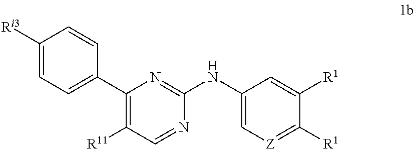

### FIELD OF THE INVENTION

This invention relates to the enzyme Janus kinase 2, or JAK2. More particularly, the invention relates to the use of JAK2 inhibitors in the treatment of multiple myeloma and related myeloproliferative neoplasms.

### BACKGROUND TO THE INVENTION

Multiple myeloma (MM) is an incurable drug resistant clonal B cell malignant neoplasm localized to the bone marrow that is associated ultimately with overproduction of monoclonal antibody by plasma cells leading to destructive lytic bone lesions and end organ damage in the form of renal and cardiac dysfunction. Current research is focused on agents that reduce activity of interleukin-6 (IL-6) which is believed to play a central role in the pathogenesis of MM through its stimulation of the JAK/STAT pathway responsible for plasma cell growth and proliferation.

Some human myeloma cell lines (HMCL) cannot proliferate or survive without exogenous IL-6 [9, 10] and some conventional drugs are ineffective in the presence of IL-6 [11-13]. The bone marrow microenvironment (BMME), known to provide supportive signals to MM cells, produces IL-6 [14] hence reducing the pro-survival effect of IL-6 may abrogate the drug resistant phenotype of MM.

Janus-activated Kinases (JAKs) are well characterized signalling kinases comprising four family members JAK1, JAK2, JAK3 and TYK2 that are important in haematological malignancy as JAK mutations have been shown to contribute to the pathogenesis of both myeloproliferative disorders [1-3] and leukaemias [4]. JAKs have an established role in signalling for many cells [reviewed by 5]. In MM, JAKs are activated by a variety of cytokines including interlukin-6 (IL-6) [6, 7], interferon-α [6, 8] and epidermal growth factor [6]. Many pathways downstream of JAKs are exploited by malignant cells.

A variety of JAK inhibitors have been developed recently, and their utility as treatments of MM is being investigated. CYT387 is a novel JAK inhibitor that can inhibit JAK1, JAK2, JAK3 and TYK2 kinase activity [15, 16]. The structure and development of the compound has recently been described [17]. Other JAK inhibitors currently in various stages of development and investigation include INCB000020 [18], INCB16562 [19], AG490 [20, 21], AZD1480 [22] and Pyridone 6 [23], as well as WP1066 [24]. Given the putative role of IL-6 in MM drug resistance JAK inhibitors are being investigated for their potential use as single agent or in combination therapy for MM. Furthermore, preliminary in vitro data has demonstrated the potential of JAK/STAT inhibition to sensitize MM cells to conventional therapies [21]. Despite these efforts, MM remains an incurable disease, resulting annually in 11,000 deaths and affecting 16,000 additional sufferers each year. It would be useful to provide additional agents and methods for the treatment of subjects afflicted with this disease.

### SUMMARY OF THE INVENTION

It has now been found that CYT 387 is active in the treatment of multiple myeloma, and particularly in treating forms of MM in which the target MM cells are CD45− and/or are IL-6 non-responsive. This effect of CYT 387 thus expands on the types of multiple myeloma that can be treated, relative to other agents that also exhibit JAK inhibition activity.

More particularly, and in one of its aspects, the present invention provides for the use of CYT 387 to inhibit growth and/or proliferation, i.e., the viability, of MM cells having a CD45− phenotype. In addition, and in another of its aspects, the present invention provides for the use of CYT387 to inhibit the growth and/or proliferation, i.e., the viability, of MM cells that are considered IL-6 non-responsive. Compounds having a JAK kinase inhibition profile like that of CYT 387 are also useful in the present method.

The activity of CYT 387 allows for the treatment of MM at a later stage of the disease, when the MM cells shift phenotypically from a CD45+ phenotype to a predominantly CD45− phenotype, thereby allowing for prolonged survival in patients desperate for treatment.

In a related aspect, the present method comprises the step of assessing the subject or a biological sample obtained therefrom, identifying multiple myeloma subjects meeting at least one of the criteria noted above, and then treating the identified subjects with CYT387 or a related compound.

In another aspect of the present invention, there is provided an article of manufacture, comprising CYT 387 or a related compound in combination with a label indicating treatment of a subject presenting with at least one of the noted criteria.

In a related aspect of the present invention, there is provided a kit comprising CYT 387 or a related compound in combination with printed instruction teaching a method of selecting a subject for CYT387 or a related compound therapy based on the selection criteria herein described.

These and other aspects and embodiments of the present invention are now described in greater detail with reference to the accompanying drawings in which:

### REFERENCE TO THE FIGURES

FIG. 1: CYT387 prevents signalling downstream of IL-6 or coculture stimulation. HMCL were incubated with or without CYT3 87 (0.5-2 μM) for 1 hour before stimulation with 10 ng/ml IL-6 for 15 minutes. Cells were then harvested and p-STAT3 (pY705) was measured. (A) By intracellular FACS with the geometric mean fluorescence intensity measured and graphed (n=3, mean±SE, Stimulated cells±CYT387 were analyzed using a One-way ANOVA with Tukey post-test *=p<0.05, **=p<0.01,***=p<0.001). (B) By western blot for p-STAT3 (pY705), total STAT3 and α-tubulin as a loading control.(C) p-STAT3 was also induced in HMCL using a direct coculture (CC) with HS5 immortalized bone marrow stromal cells or primary bone marrow stromal cells or a transwell (TW) “soluble only” CC with HS5. HMCL were fluorescently labelled with CD38 or CD138 and stimulated for 15 minutes with or without co-treatment with 2 μM CYT387. Representative plots of NCI-H929, (n=3, for NCI-H929, OCI-MY1 and U266). (D) NCI-H929, OCI-MY1 and U266 cells were starved overnight and stimulated with 5 ng/ml IL-6 and 100 ng/ml IGF-1 for 15 minutes with or without co-treatment with 2 μM CYT387. p-AKT (pS473) and p-ERK1/2 (pT202/pY204) were measured by intracellular FACS with the geometric mean fluorescence intensity normalized to the untreated (UT) control and averaged (n=4, mean±SE. Stimulated cells±CYT387 were analyzed using a One-way ANOVA with Tukey post-test *=p<0.05, **=p<0.01).

FIG. 2: CYT387 inhibits HMCL proliferation. (A) CYT387 inhibits HMCL in a time and dose dependent manner. IL-6 phenotype HMCL (ANBL-6, OCIMY1,U266 and XG-1) and non-IL-6 phenotype HMCL (LP-1, NCI-H929, OPM2 and RPMI-8226) were cultured for 24, 48 and 72 hours UT, with CYT387 (0.1, 0.5, 1, 2.5 or 5 μM) or with vehicle (DMSO). Cell proliferation was then determined by MTS Assay (72 hour data shown, n=3, mean±SE) (B) Treatment with CYT387 inhibits myeloma cell proliferation even in the presence of IL-6. Absolute cell numbers of viable cells were determined by haemocytometer counts of HMCL cultured alone (Untreated) with IL-6 (10 ng/ml) with CYT387 (0.5-1 μM) or with IL-6 and CYT387. Culture with CYT387 greatly decreased the proliferation of the HMCL over 72 hours (treatment at time 0 only). Results represent the mean of 3 independent experiments±SE. (C) CYT3 87 prevents cell cycling. HMCL were treated with CYT3 87 (1 μM or 5 μM) for 24 and 72 hours then they were harvested and fixed and cell cycle analysed by FACS. Representative cell cycle plots of CIH929 UT or 5 μM CYT387 for 24 or 72 hours, with mean of 4 independent experiments±SE of cycling cells in G2/M phase of the cell cycle.

FIG. 3: CYT387 induces apoptosis in HMCL. (A) Representative Annexin-V and Propidium Iodide (PI) plots of NCI-H929. UT, Vehicle (DMSO) treated, 24 hours 5 μM CYT387 treatment and 72 hours 5 μM CYT387 treatment. (B) Proportion of viable (Annexin-V- and PI-) cells after CYT387 treatment compared to UT. Data shown is the mean of 4 independent experiments±SE.

FIG. 4: CYT387 synergizes with melphalan and bortezomib in HMCL. (A) Dose effect curves of NCI-H929, OCI-MY1 and U266 after 24 and 48 hours treatment CYT387 treatment (0.5-10 μM) as determined by the proportion of PI+ cells minus background death (untreated). Mean of 4 independent experiments±SE. (B) CYT387 in combination with melphalan or bortezomib show synergism. Synergy was measured using a combination index calculated by Calcusyn software, where values less than 1 represent synergism, plotted against the fraction of cells killed with various dose/ratios of the drugs. Synergy is seen between melphalan and CYT387 at a range of doses/ratios/cell lines and time points. Bortezomib and CYT387 demonstrated synergistic or nearly additive in 18/24 combinations. Synergism was calculated from dose effect curves of the mean of 4 independent experiments.

FIG. 5: CYT387 induces apoptosis in primary samples as a single agent or in combination with melphalan and bortezomib. (A) Proportion of apoptotic (Apo 2.7+) CD38+ CD45− primary patient myeloma cells after 48 hours CYT387 treatment (n=6). (B) Synergy between CYT387 and melphalan or bortezomib after 24 hours treatment as determined by calcusyn software in primary patient CD38+ CD45− cells.

### DETAILED DESCRIPTION OF THE INVENTION

CYT 387 is a phenylaminopyrimidine compound having CAS registration number CAS 1056634-68-4, the chemical name N-(cyanomethyl)-4-[2-[[4-(4-morpholinyl)phenyl]amino]-4-pyrimidinyl]-benzamide, and the structure shown below:

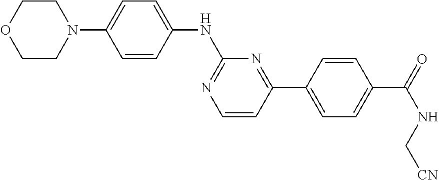 [SMILES: N#CCNC(=O)c1ccc(-c2ccnc(Nc3ccc(N4CCOCC4)cc3)n2)cc1]

Synthesis, formulation and therapeutic use of CYT 387 is described in WO 2008/109943 published 18 Sep. 2008; and in Blood, 2010, 115(25):5232-40. Of course, CYT387 can be used in the form of a salt, solvate or prodrug if desired.

“Related compounds” are compounds related to CYT 387 by their selective JAK inhibition signature, in which a preference is shown for binding to and inhibition of JAK2 and JAK1, relative to JAK3 and other members of the kinase family, and by their structural conformance to the formula:

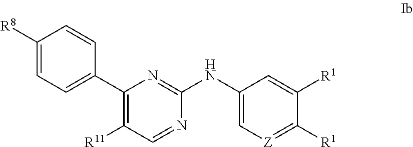

wherein

Z is independently selected from N and CH;

R¹ is independently selected from H, halogen, OH, CONHR², CON(R²)₂, CF₃, R²OR², CN, morpholino, thiomorpholinyl, thiomorpholino-1,1-dioxide, substituted or unsubstituted piperidinyl, substituted or unsubstituted piperazinyl, imidazolyl, substituted or unsubstituted pyrrolidinyl and C₁₋₄alkylene wherein the carbon atoms are optionally replaced with NR^{Y} and/or O substituted with morpholino, thiomorpholinyl, thiomorpholino-1,1-dioxide, substituted or unsubstituted piperidinyl, substituted or unsubstituted piperazinyl, imidazolyl or substituted or unsubstituted pyrrolidinyl;

R² is substituted or unsubstituted C₁₋₄alkyl;

R^{Y} is H or substituted or unsubstituted C₁₋₄alkyl;

R⁸ is R^{X}CN;

R^{X} is substituted or unsubstituted C₁₋₄alkylene wherein up to 2 carbon atoms can be optionally replaced with CO, NSO₂R¹, NR^{Y}, CONR^{Y}, SO, SO₂ or O;

R¹¹ is H or C₁₋₄alkyl,

or an enantiomer thereof, a prodrug thereof or a pharmaceutically acceptable salt thereof.

The term “C₁₋₄alkyl” refers to straight chain or branched chain hydrocarbon groups having from 1 to 4 carbon atoms. Examples include methyl, ethyl, propyl, isopropyl, butyl, isobutyl, sec-butyl, and tert-butyl.

The term “halogen” refers to fluorine, chlorine, bromine and iodine.

The term “substituted” refers to a group that is substituted with one or more groups selected from C₁₋₄ alkyl, C₃₋₆ cycloalkyl, C₂₋₆ alkenyl, C₂₋₆ alkynyl, C₁₋₆ alkylaryl, aryl, heterocycylyl, halo, haloC₁₋₆alkyl, haloC₃₋₆cycloalkyl, haloC₂₋₆alkenyl, haloC₂₋₆alkynyl, haloaryl, haloheterocycylyl, hydroxy, C₁₋₆ alkoxy, C₂₋₆alkenyloxy, C₂₋₆alkynyloxy, aryloxy, heterocyclyloxy, carboxy, haloC₁₋₆alkoxy, haloC₂₋₆alkenyloxy, haloC₂₋₆alknyloxy, haloaryloxy, nitro, nitroC₁₋₆alkyl, nitroC₂₋₆alkenyl, nitroaryl, nitroheterocyclyl, azido, amino, C₁₋₆alkylamino, C₂₋₆alkenylamino, C₂₋₆alkynylamino, arylamino, heterocyclamino acyl, C₁₋₆alkylacyl, C₂₋₆alkenylacyl, C₂₋₆alkynylacyl, arylacyl, heterocycylylacyl, acylamino, acyloxy, aldehydo, C₁₋₆alkylsulphonyl, arylsulphonyl, C₁₋₆alkylsulphonylamino, arylsulphonylamino, C₁₋₆alkylsulphonyloxy, arylsulphonyloxy, C₁₋₆alkylsulphenyl, C₂₋₆alklysulphenyl,arylsulphenyl, carboalkoxy, carboaryloxy, mercapto, C₁₋₆alkylthio, arylthio, acylthio, cyano and the like. Preferred substituents are selected from the group consisting of C₁₋₄ alkyl, C₃₋₆ cycloalkyl, C₂₋₆ alkenyl, C₂₋₆ alkynyl, C₁₋₆ alkylaryl, aryl, heterocycylyl, halo, haloaryl, haloheterocycylyl, hydroxy, C₁₋₄ alkoxy, aryloxy, carboxy, amino, C₁₋₆alkylacyl, arylacyl, heterocycylylacyl, acylamino, acyloxy, C₁₋₆alkylsulphenyl, arylsulphonyl and cyano.

The term “aryl” refers to single, polynuclear, conjugated or fused residues of aromatic hydrocarbons. Examples include phenyl, biphenyl, terphenyl, quaterphenyl, naphthyl, tetrahydronaphthyl, anthracenyl, dihydroanthracenyl, benzanthracenyl, dibenxanthracenyl and phenanthrenyl.

The term “unsaturated N-containing 5 or 6-membered heterocyclyl” refers to unsaturated, cyclic hydrocarbon groups containing at least one nitrogen.

Suitable N-containing heterocyclic groups include unsaturated 5 to 6-membered heteromonocyclic groups containing 1 to 4 nitrogen atoms, for example, pyrrolyl, imidazolyl, pyrazolyl, pyridyl, pyrimidinyl, pyrazinyl, pyridazinyl, triazolyl or tetrazolyl; unsaturated 5 or 6-membered heteromonocyclic group containing 1 to 2 oxygen atoms and 1 to 3 nitrogen atoms, such as, oxazolyl, isoxazolyl or oxadiazolyl; and unsaturated 5 or 6-membered heteromonocyclic group containing 1 to 2 sulphur atoms and 1 to 3 nitrogen atoms, such as, thiazolyl or thiadiazolyl.

In preferred embodiments, compounds related to CYT 387 include those in which R¹ is substituted in the para position by morpholinyl and in the ortho position by H, Z is carbon, and R¹¹ is H, methyl or methoxy.

In particularly preferred embodiments, R⁸ is —C(O)—NH—CH₂—CH═N; —C(O)—NH—C(CH₃)₂CH═N; or —NH—C(O)—CH₂—CH═N.

Specific compounds related to CYT 387 useful in accordance with the present method include:
N-(cyanomethyl)-4-(2-(4-morpholinophenylamino)pyrimidin-4-yl)benzamide;
N-(cyanomethyl)-3-(2-(4-morpholinophenylamino)pyrimidin-4-yl)benzamide;
N-(cyanomethyl)-3-methyl-4-(2-(4-morpholinophenylamino)pyrimidin-4-yl)benzamide;
N-(cyanomethyl)-2-methyl-4-(2-(4-morpholinophenylamino)pyrimidin-4-yl)benzamide;
2-cyano-N-(3-(2-(4-morpholinophenylamino)pyrimidin-4-yl)benzyl)acetamide;
2-cyano-N-(3-(2-(4-morpholinophenylamino)pyrimidin-4-yl)phenyl)acetamide;
N-(cyanomethyl)-4-(2-(3-morpholinophenylamino)pyrimidin-4-yl)benzamide;
N-(cyanomethyl)-4-(2-(4-thiomorpholinophenylamino)pyrimidin-4-yl)benzamide; and
N-(cyanomethyl)-4-(2-(4-(morpholinomethyl)phenylamino)pyrimidin-4-yl)benzamide.

In the present invention, CYT 387 and related compounds are used to treat multiple myeloma (MM) cells that have a CD45 negative (CD45−) phenotype, and/or MM cells that are considered IL-6 non-responsive. MM cells are the disease cells that form plasmacytoma tumours that are the hallmark of multiple myeloma. “CD45− phenotype” refers to a MM cell that tests negative or dim, as distinct from intermediate to bright, for surface expression of the protein marker known as CD45, which is a well-known marker of all hematopoietic cells. The CD45− phenotype is also ascribed herein with reference to a population of MM cells in which the prevalence of CD45− cells within that population exceeds at least about 10% of that population, such as at least about 15%, or 20%, or 25%, or 30%, or 35%, or 40%, or 45% or at least about 50% of that population. Detection of CD45 on the cellular surface is readily achieved using fluorescence-labeled CD45 monoclonal antibody and established techniques of fluorescence-activation cell sorting (FACS) or any related means for identifying cells that bind the CD45 antibody. Reference can be made for instance to the articles published by Moreau et al, Haematologica, 2004, 89(5):547, and by Kumar et al, Leukemia, 2005, 19:1466, the disclosures of which are incorporated herein by reference.

MM cells that are “IL-6 non-responsive” are identified as cells that do not rely for survival on the presence of interleukin-6 (IL-6). Thus, a MM cell that is IL-6 non-responsive shows insubstantial response, in terms such as IL-6 receptor stimulation or downstream signalling events, when incubated with an otherwise stimulatory amount of IL-6. Such MM cells can particularly include those MM cells that are resident in the bone marrow environment, and which thus grow in the same environment as bone marrow stromal cells, but they also include MM cells in circulation that are not exposed to the marrow environment.

Within the realm of MM and its progression and development, CD45 represents an early marker of the disease MM cells. As the disease progresses, a shift occurs in CD45 phenotype of those cells, in which the predominance of CD45+ cells wanes, and the population of disease plasma cells becomes predominantly CD45− (see Kumar et al, Leukemia, 2005, 19(8):1466). A shift also occurs in the number of IL-6 non-responsive cells, with this cell form becoming predominant in the later stages of disease.

In the present method, the use of JAK inhibitors is proposed for the treatment of MM cells, and plasmacytoma tumours that arise therefrom, that have acquired the CD45− and/or IL-6 non-responsive phenotype. The effect of JAK inhibition on this particular cell population is surprising, given that the JAK2 response to stimulation of the IL-6 receptor is believed to have a central and important role in progression of MM. Even when this IL-6 pathway is not involved in MM disease progression, CYT 387 functions to inhibit the growth and/or proliferation of these cells.

“Related disorders” are disorders related to MM as plasma cell disorders characterized by a clonal population of hematopoietic B cells that produce a monoclonal protein (M protein, or paraprotein). The clinical manifestations of these disorders result from the uncontrolled and progressive proliferation of a plasma cell clone, the effect of normal bone marrow replacement, and the overproduction of monoclonal proteins. MM is a plasma cell dyscrasia and includes newly diagnosed as well as relapsed MM.

Subjects, most notably human patients, who present with MM are identifiable using any of the established diagnostic criteria and staging parameters. These include criteria established by the International Myeloma Workshop which distinguishes symptomatic MM subjects from those having asymptomatic MM or gammopathy of undetermined significance (MGUS), by the presence of M protein in serum or urine, clonal bone marrow plasmacytosis or plasmacytoma and related organ and tissue impairment. For staging of MM, the guidelines proposed by the Southwest Oncology Group (SWOG) can be used, which rely essentially on measurements of B2-microglobulin and the relative presence of serum albumin, with >5.5 mg/L β2-M and <3.0 g/dL indicating stage IV disease. Other useful guidelines have been established using the Duire and Salmon staging system (using hemoglobin, serum calcium, radiography and M protein).

In the present method, subjects selected for treatment are those presenting with MM that also display an increase in the presence of CD45− cells within the population of MM cells. The increase in the presence of the CD45− cells is seen in subjects having newly diagnosed or relapsed MM, relative to subjects afflicted with smoldering MM or MGUS. Subjects having a relatively dramatic increase in the prevalence of CD45− MM cells particularly include those MM subjects diagnosed with stage III or stage IV of the disease. In a preferred embodiment, the number of CD45− cells within the population is at least 10% of the total population, such as 15%, 20%, 25%, 30%, 35%, 40%, 45%, 50% or more of the total MM cell population. Thus, in a representative sample of 100 MM cells extracted from the subject, the patient population targeted for treatment by the present method includes those presenting with a MM cell population in which 10-50% or more of the MM cells test negative for the CD45 marker.

In the present method, subjects diagnosed with MM can first be screened to select those subjects presenting with a MM cell population in which the CD45− phenotype is increased relative to subjects afflicted with early stages of the disease, such as the smoldering MM stage. Screening is achieved using a MM cell population extracted from the subject, and then assaying the population such as by CD45 MAb-based flow cytometry to identify subjects in which there is an increase in CD45− MM cells. The MM cell population can also be assessed to reveal the prevalence of IL-6 non-responsive cells, the presence of which indicates the subject is a candidate for treatment by the present method.

For use in the present method, CYT 387 or a related compound is formulated according to standard pharmaceutical practice.

The compounds may be prepared as salts which are pharmaceutically acceptable, such as salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, trihalomethanesulfonic, toluenesulfonic, benzenesulfonic, isethionic, salicylic, sulphanilic, aspartic, glutamic, edetic, stearic, palmitic, oleic, lauric, pantothenic, tannic, ascorbic, valeric and orotic acids. Salts of amine groups may also comprise quaternary ammonium salts in which the amino nitrogen atom carries a suitable organic group such as an alkyl, alkenyl, alkynyl or aralkyl moiety.

Where a compound possesses a chiral center the compound can be used as a purified enantiomer or diastereomer, or as a mixture of any ratio of stereoisomers. It is however preferred that the mixture comprises at least 70%, 80%, 90%, 95%, 97.5% or 99% of the preferred isomer, where the preferred isomer gives the desired level of potency and selectivity.

Prodrugs of the compounds of formula Ib can also be administered. For example, compounds of formula Ib having free amino, amido, hydroxy or carboxylic acid groups can be converted into prodrugs. Prodrugs include compounds wherein an amino acid residue, or a polypeptide chain of two or more (e.g., two, three or four) amino acid residues which are covalently joined through peptide bonds to free amino, hydroxy and carboxylic acid groups of compounds of the invention. The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methioine sulfone. Prodrugs also include compounds wherein carbonates, carbamates, amides and alkyl esters which are covalently bonded to the above substituents of compounds of the present invention through the carbonyl carbon prodrug sidechain. Prodrugs also include phosphate derivatives of compounds (such as acids, salts of acids, or esters) joined through a phosphorus-oxygen bond to a free hydroxyl of compounds of formula Ib. Prodrugs may also include N-oxides, and S-oxides of appropriate nitrogen and sulfur atoms in formula Ib.

The compound may be administered as a pharmaceutical composition comprising at least one of the compounds of the formula Ib and a pharmaceutically acceptable carrier. The carrier must be “pharmaceutically acceptable” means that it is compatible with the other ingredients of the composition and is not deleterious to a subject. The compositions may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavours, etc.) according to techniques such as those well known in the art of pharmaceutical formulation (See, for example, Remington: The Science and Practice of Pharmacy, 21st Ed., 2005, Lippincott Williams & Wilkins).

The compound may be administered by any suitable means, for example, orally, such as in the form of tablets, capsules, granules or powders; sublingually; buccally; parenterally, such as by subcutaneous, intravenous, intramuscular, intra(trans)dermal, or intracisternal injection or infusion techniques (e.g., as sterile injectable aqueous or non-aqueous solutions or suspensions); nasally such as by inhalation spray or insufflation; topically, such as in the form of a cream or ointment ocularly in the form of a solution or suspension; vaginally in the form of pessaries, tampons or creams; or rectally such as in the form of suppositories; in dosage unit formulations containing non-toxic, pharmaceutically acceptable vehicles or diluents. The compounds may, for example, be administered in a form suitable for immediate release or extended release. Immediate release or extended release may be achieved by the use of suitable pharmaceutical compositions comprising the compounds, or, particularly in the case of extended release, by the use of devices such as subcutaneous implants or osmotic pumps.

The pharmaceutical compositions for the administration may conveniently be presented in dosage unit form and may be prepared by any of the methods well known in the art of pharmacy. These methods generally include the step of bringing the compound of formula Ib into association with the carrier which constitutes one or more accessory ingredients. In general, the pharmaceutical compositions are prepared by uniformly and intimately bringing the compound into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents such as sweetening agents, flavouring agents, colouring agents and preserving agents, e.g. to provide pharmaceutically stable and palatable preparations. Tablets contain the compound of formula Ib in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the compound of formula Ib is mixed with an inert solid diluent, for example, calcium carbonate, calcium phosphate or kaolin, or as soft gelatin capsules wherein the compound of formula Ib is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the compound of formula Ib in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectable formulations.

For administration to the respiratory tract, including intranasal administration, the active compound may be administered by any of the methods and formulations employed in the art for administration to the respiratory tract.

Thus in general the active compound may be administered in the form of a solution or a suspension or as a dry powder.

Solutions and suspensions will generally be aqueous, for example prepared from water alone (for example sterile or pyrogen-free water) or water and a physiologically acceptable co-solvent (for example ethanol, propylene glycol or polyethylene glycols such as PEG 400).

Such solutions or suspensions may additionally contain other excipients for example preservatives (such as benzalkonium chloride), solubilizing agents/surfactants such as polysorbates (eg. Tween 80, Span 80, benzalkonium chloride), buffering agents, isotonicity-adjusting agents (for example sodium chloride), absorption enhancers and viscosity enhancers. Suspensions may additionally contain suspending agents (for example microcrystalline cellulose and carboxymethyl cellulose sodium).

Solutions or suspensions are applied directly to the nasal cavity by conventional means, for example with a dropper, pipette or spray. The formulations may be provided in single or multidose form. In the latter case a means of dose metering is desirably provided. In the case of a dropper or pipette this may be achieved by the subject administering an appropriate, predetermined volume of the solution or suspension. In the case of a spray this may be achieved for example by means of a metering atomising spray pump.

Administration to the respiratory tract may also be achieved by means of an aerosol formulation in which the compound is provided in a pressurized pack with a suitable propellant, such as a chlorofluorocarbon (CFC), for example dichlorodifluoromethane, trichlorofluoromethane or dichlorotetrafluoroethane, carbon dioxide or other suitable gas. The aerosol may conveniently also contain a surfactant such as lecithin. The dose of active compound may be controlled by provision of a metered valve.

Alternatively the active compound may be provided in the form of a dry powder, for example a powder mix of the compound in a suitable powder base such as lactose, starch, starch derivatives such as hydroxypropylmethyl cellulose and polyvinylpyrrolidine (PVP). Conveniently the powder carrier will form a gel in the nasal cavity. The powder composition may be presented in unit dose form, for example in capsules or cartridges of eg. gelatin, or blister packs from which the powder may be administered by means of an inhaler.

In formulations intended for administration to the respiratory tract, including intranasal formulations, the active compound will generally have a small particle size, for example of the order of 5 microns or less. Such a particle size may be obtained by means known in the art, for example by micronisation.

When desired, formulations adapted to give sustained release of the active compound may be employed.

The active compound may be administered by oral inhalation as a free-flow powder via a “Diskhaler” (trade mark of Glaxo Group Ltd) or a meter dose aerosol inhaler.

The compound may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions suitable for vaginal administration may be presented as pessaries, tampons, creams, gels, pastes, foams or sprays containing in addition to the active ingredient such carriers as are known in the art to be appropriate.

For topical use, creams, ointments, jellies, solutions or suspensions, etc., containing the compound are employed. For purposes of this application, topical application can include mouthwashes and gargles.

For application to the eye, the active compound may be in the form of a solution or suspension in a suitable sterile aqueous or non-aqueous vehicle. Additives, for instance buffers, preservatives including bactericidal and fungicidal agents, such as phenyl mercuric acetate or nitrate, benzalkonium chloride, or chlorohexidine and thickening agents such as hypromellose may also be included.

The compound can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolisable lipid capable of forming liposomes can be used. The present compositions in liposome form can contain, in addition to a compound of the present invention, stabilizers, preservatives, excipients and the like. The preferred lipids are the phospholipids and phosphatidyl cholines, both natural and synthetic. Methods to form liposomes are known in the art.

The compound may also be presented for use in the form of veterinary compositions, which may be prepared, for example, by methods that are conventional in the art. Examples of such veterinary compositions include those adapted for:

(a) oral administration, external application, for example drenches (e.g. aqueous or non-aqueous solutions or suspensions); tablets or boluses; powders, granules or pellets for admixture with feed stuffs; pastes for application to the tongue;

(b) parenteral administration for example by subcutaneous, intramuscular or intravenous injection, e.g. as a sterile solution or suspension; or (when appropriate) by intramammary injection where a suspension or solution is introduced in the udder via the teat;

(c) topical applications, e.g. as a cream, ointment or spray applied to the skin; or

(d) rectally or intravaginally, e.g. as a pessary, cream or foam.

The pharmaceutical composition may further comprise other therapeutically active compounds as noted herein which are usually applied in the treatment of the above mentioned pathological conditions. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Examples of other therapeutic agents include the following: endothelin receptor antagonists (eg ambrisentan, bosentan, sitaxsentan), PDE-V inhibitors (eg sildenafil, tadalafil, vardenafil), Calcium channel blockers (eg amlodipine, felodipine, varepamil, diltiazem, menthol), prostacyclin, treprostinil, iloprost, beraprost, nitric oxide, oxygen, heparin, warfarin, diuretics, digoxin, cyclosporins (e.g., cyclosporin A), CTLA4 Ig, antibodies such as ICAM 3, anti-IL 2 receptor (Anti Tac), anti CD45RB, anti CD2, anti CD3 (OKT 3), anti CD4, anti CD80, anti CD86, agents blocking the interaction between CD40 and gp39, such as antibodies specific for CD40 and/or gp39 (i.e., CD154), fusion proteins constructed from CD40 and gp39 (CD401g and CD8gp39), inhibitors, such as nuclear translocation inhibitors, of NF kappa B function, such as deoxyspergualin (DSG), cholesterol biosynthesis inhibitors such as HMG CoA reductase inhibitors (lovastatin and simvastatin), non-steroidal anti-inflammatory drugs (NSAIDs) such as ibuprofen, aspirin, acetaminophen, leflunomide, deoxyspergualin, cyclooxygenase inhibitors such as celecoxib, steroids such as prednisolone or dexamethasone, gold compounds, beta-agonists such as salbutamol, LABA's such as salmeterol, leukotriene antagonists such as montelukast, antiproliferative agents such as methotrexate, FK506 (tacrolimus, Prograf), mycophenolate mofetil, cytotoxic drugs such as azathioprine, VP-16, etoposide, fludarabine, doxorubin, adriamycin, amsacrine, camptothecin, cytarabine, gemcitabine, fluorodeoxyuridine, melphalan and cyclophosphamide, antimetabolites such as methotrexate, topoisomerase inhibitors such as camptothecin, DNA alkylators such as cisplatin, kinase inhibitors such as sorafenib, microtubule poisons such as paclitaxel, TNF-α inhibitors such as tenidap, anti-TNF antibodies or soluble TNF receptor, hydroxy urea and rapamycin (sirolimus or Rapamune) or derivatives thereof.

In one embodiment, MM subjects are treated with a combination of CYT 387 or a related compound, and melphalan.

In another embodiment, MM subjects are treated with a combination of CYT 387 or a related compound, and bortezomib.

When other therapeutic agents are employed in combination with the compounds of the present invention they may be used for example in amounts as noted in the Physician Desk Reference (PDR) or as otherwise determined by one of ordinary skill in the art.

In a preferred embodiment, the present method utilizes CYT 387 in combination with a compound selected from melphalan and bortezomib.

The present invention also provides both an article of manufacture and a kit, comprising a container comprising CYT387 or a related compound in an amount effective to treat MM. The container may be simply a bottle comprising the compound in oral dosage form, each dosage form comprising a unit dose of the compound, in an amount for instance from about 50 mg to 400 mg, such as 200 mg or 300 mg. The kit will further comprise printed instructions teaching the present method of selecting subjects for treatment. The article of manufacture will comprise a label or the like, indicating treatment of a subject according to the present method of patient selection.

Generally, the term “treatment” means affecting a subject, tissue or cell to obtain a desired pharmacological and/or physiological effect and include: (a) preventing the disease from occurring in a subject that may be predisposed to the disease, but has not yet been diagnosed as having it; (b) inhibiting the disease, i.e., arresting its development; or (c) relieving or ameliorating the effects of the disease, i.e., cause regression of the effects of the disease. In one embodiment, treatment achieves the result of reducing the number of CD45− and/or IL-6 non-responsive MM cells in the recipient subject.

The term “subject” refers to any animal having a disease which requires treatment by the present method. In addition to primates, such as humans, a variety of other mammals can be treated using the methods of the present invention. For instance, mammals including, but not limited to, cows, sheep, goats, horses, dogs, cats, guinea pigs, rats or other bovine, ovine, equine, canine, feline, rodent or murine species can be treated. Dogs in particular are known to experience multiple myeloma.

The term “administering” should be understood to mean providing a compound of the invention to a subject in need of treatment.

The term “therapeutically effective amount” refers to the amount of the compound that will elicit the biological or medical response of a tissue, system, animal or human that is being sought by the researcher, veterinarian, medical doctor or other clinician.

In the treatment or prevention of multiple myeloma, an appropriate compound dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient. The dosage may be selected, for example to any dose within any of these ranges, for therapeutic efficacy and/or symptomatic adjustment of the dosage to the patient to be treated. The compound will preferably be administered on a regimen of 1 to 4 times per day, preferably once or twice per day.

It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In order to exemplify the nature of the present invention such that it may be more clearly understood, the following non-limiting examples are provided.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

CYT387 is an inhibitor of the kinase enzymes JAK1 and JAK2, which have been implicated in a family of hematological conditions known as myeloproliferative neoplasms, including myelofibrosis, and as well in numerous disorders including indications in hematology, oncology and inflammatory diseases. Myelofibrosis is a chronic debilitating disease in which a patient's bone marrow is replaced by scar tissue and for which treatment options are limited or unsatisfactory.

### Synthesis of CYT 387

A mixture of 4-ethoxycarbonylphenyl boronic acid (23.11 g, 119 mmol), 2,4-dichloropyrimidine (16.90 g, 113 mmol), toluene (230 mL) and aqueous sodium carbonate (2 M, 56 mL) was stirred vigorously and nitrogen was bubbled through the suspension for 15 minutes. Tetrakis(triphenylphosphine)palladium[0] (2.61 g, 2.26 mmol) was added. Nitrogen was bubbled through for another 10 min., the mixture was heated to 100° C., then at 75° C. overnight. The mixture was cooled, diluted with ethyl acetate (200 mL), water (100 mL) was added and the layers were separated. The aqueous layer was extracted with ethyl acetate (100 ml) and the two organic extracts were combined. The organics were washed with brine, filtered through sodium sulfate, concentrated, and the resultant solid was triturated with methanol (100 mL) and filtered. The solids were washed with methanol (2×30 mL) and air dried. This material was dissolved in acetonitrile (150 mL) and dichloromethane (200 mL), stirred with MP.TMT Pd-scavenging resin (Agronaut part number 800471) (7.5 g) over 2 days. The solution was filtered, the solids were washed with dichloromethane (2×100 mL), and the filtrate concentrated to give ethyl 4-(2-chloropyrimidin-4-yl)benzoate as an off-white solid (17.73 g, 60%)—additional washing with dichloromethane yielded a further 1.38 g and 0.5 g of product.

A mixture of ethyl 4-(2-chloropyrimidin-4-yl)benzoate (26.15 g, 99.7 mmol) and 4-morpholinoaniline (23.10 g, 129.6 mmol) was suspended in 1,4-dioxane (250 mL). p-Toluenesulfonic acid monohydrate (17.07 g, 89.73 mmol) was added. The mixture was heated at reflux for 40 h., cooled to ambient temperature, concentrated then the residue was partitioned between ethyl acetate and 1:1 saturated sodium bicarbonate/water (1 L total). The organic phase was washed with water (2×100 mL) and concentrated. The aqueous phase was extracted with dichloromethane (3×200 mL). The material which precipitated during this workup was collected by filtration and set aside. The liquid organics were combined, concentrated, triturated with methanol (200 mL) and filtered to yield additional yellow solid. The solids were combined, suspended in methanol (500 mL), allowed to stand overnight then sonicated and filtered. The solids were washed with methanol (2×50 mL) to give, after drying, ethyl 4-(2-(4-morphonlinophenylamino)pyrimidin-4-yl)benzoate (35.39 g, 88%).

A solution of ethyl 4-(2-(4-morpholinophenylamino)pyrimidin-4-yl)benzoate (35.39 g, 87.6 mmol) in 3:1 methanol/tetrahydrofuran (350 mL) was treated with lithium hydroxide (4.41 g, 183.9 mmol) in water (90 mL). The mixture was heated at reflux for 2 h., cooled, concentrated and acidified with hydrochloric acid (2M, 92.5 mL, 185 mmol) The dark precipitate was filtered, washed with water, and dried under vacuum. The solid was ground to a powder with a mortar and pestle, triturated with methanol (500 mL) then filtered again to yield 4-(2-(4-morpholinophenylamino)pyrimidin-4-yl)benzoic acid as a muddy solid. This material was washed with ether, air dried overnight, and ground to a fine powder with mortar and pestle. On the basis of mass recovery (34.49 g) the yield was assumed to be quantitative.

To a suspension of 4-(2-(4-morpholinophenylamino)pyrimidin-4-yl)benzoic acid (theoretically 32.59 g, 86.6 mmol) in DMF (400 mL) was added triethylamine (72.4 mL, 519.6 mmol, 6 eq.) The mixture was sonicated to ensure dissolution Aminoacetonitrile hydrochloride (16.02 g, 173.2 mmol) was added followed by N-hydroxybenzotriazole (anhydrous, 14.04 g, 103.8 mmol) and 1-ethyl-3-(dimethylaminopropyl)carbodiimide hydrochloride (19.92 g, 103.8 mmol). The suspension was stirred vigorously overnight. The solvent was evaporated under reduced pressure, the residue was diluted with 5% sodium bicarbonate (400 mL) and water (300 mL), giving a yellow solid, which was broken up and filtered. The solids were washed several times with 100 mL portions of water, triturated with hot methanol/dichloromethane (500 mL, 1:1), concentrated to a volume of approximately 300 mL), cooled and filtered. The solids were washed with cold methanol (3×100 mL), ether (200 mL) and hexane (200 mL) prior to drying to afford CYT 387 (31.69 g, 88%). M.p. 238-243° C.

Synthesis of related compounds is described by Bums et al, in Bioorganic & Medicinal Chemistry Letters, 2009, 19:5887 and in WO2008/109943, both disclosures being incorporated herein by reference.

### Materials and Methods

### Reagents

The JAK1/2 inhibitor CYT387 was dissolved in DMSO. The proteasome inhibitor bortezomib (Janssen-Cilag) was reconstituted in saline. The alkylating agent melphalan (Sigma was dissolved in 0.5% HCl.EtOH. All stock drug solutions were diluted in complete RPMI-1640 culture medium to various concentrations for experimentation.

### Cell Lines and Culture Conditions

HMCL LP-1, NCI-H929, OPM2, RPMI-8226 and U266 and the human stromal cell line HS5 were obtained from the American Type Culture Collection, USA. ANBL6, OCI-MY1 and XG-1 were a kind gift from the Winthrop P Rockefeller Cancer Institute, Arkansas. HMCL were grown and treated at densities between 2.0 and 5.0×105 cell/ml in RPMI-1640 media (Gibco, Invitrogen) supplemented with 10% heat inactivated foetal bovine serum (FBS, Lonza) and 2 mM Lgiutamine (Gibco, Invitrogen), IL-6 dependent cells lines were cultured with 2-5 ng/ml IL-6 as required. All cells were cultured in a humidified incubator at 37° C. with 5% CO2. All HMCL were passaged 24 hours prior to experimental set-up to ensure high viability and cycling.

### Primary Samples

Primary MM samples were obtained from Bone Marrow Aspirates from relapsed and refractory MM patients following written informed consent with approval from the Alfred Hospital Research and Ethics Committee and isolated and treated as previously described [25]. Briefly, bone marrow mononuclear cells (BMMC) were isolated with Ficoll-Paque Plus (Amersham Biosciences), washed in PBS and red blood cells were lysed with NH4Cl solution (8.29 g/L ammonium chloride, 0.037 g/L EDTA, 1 g/L potassium bicarbonate). Cells were then washed again in PBS and quantitated by haemocytometer. BMMC samples were then cultured in complete RPMI-1640 media (as above for HMCL) for 24 hours. Subsequently the BMMC were plated at 5×105 cells/ml and were treated with CYT387 (5-50 μM) alone or (dependant on cell numbers) in combination with bortezoniib (5-40 nM) or melphalan (50-200 μM) for 24 and/or 48 hours. Drug-induced MM specific cell apoptosis was then compared to untreated and vehicle controls by staining for CD45 FITC (BD), CD38 PerCP-Cy5.5 (BD) and Apo 2.7 PE (Immunotech) to determine apoptosis in CD45−CD38+ MM cells. Samples were subsequently analyzed by FACS. Primary bone marrow stromal cells (BMSC) were also collected from patient BMMC, cells that adhered to the flask after an initial 24 hour culture were cultured with continued selection for adherent cells over several passages. Once cells had expanded in culture they were used in coculture (CC) to stimulate MM cells in parallel to experiments utilising the HS5 stromal cell line.

### Western Blots (WB)

HMCL were treated with CYT387 (1 or 2 μM) for 60 minutes and then stimulated with 10 ng/ml IL-6 for 15 minutes. Protein lysates of CYT387 treated and untreated HMCL were made with RIPA Buffer (50 mM Tris.HCl pH 7.4, 150 mM NaCl, 1 mM PMSF, 1 mM EDTA, 5 μg/ml Aprotinin, 5 μg/ml Leupeptin, 1% Triton X-100, 1% Sodium deoxycholate and 0.1% SDS). Briefly cells were incubated in RIPA buffer on ice for 30-60 minutes before being centrifuged at 16,100×g for 20 minutes at 4° C. and the supernatant collected. Protein concentration was quantified using DC Protein Assay (Bio-Rad) as per manufacturer's instructions. Subsequently, 100 μg of each protein lysate was separated by 10% SDS-PAGE and blotted onto nitrocellulose (Hybond ECL, Amersham) using the Bio-Rad semi-dry transfer system. Membranes were blocked with 5% skim milk powder 0.1% Tween-20/PBS for 60 minutes then incubated with mouse monoclonal antiphospho-STAT3 (pY705, Santa Cruz), mouse monoclonal anti-STAT3 (Santa Cruz) or mouse monoclonal anti-a-tubulin (Sigma-Aldrich) for 1-2 hours at room temperature or overnight at 4° C. The blots were washed three times for 15 minutes in 0.1% Tween-20/PBS, then incubated with secondary HRP lagged antibody (swine anti-rabbit Ig HRP (Dako) or rabbit anti-mouse Ig HRP (Dako)) for 1-2 hours at room temperature before washing as above. Blots were visualized with Supersignal west pico ECL reagents (Pierce).

### Intracellular FACS

Activation of the JAK/STAT, PI3K/AKT and Ras/MAPK pathways was investigated using intracellular flow cytometry to measure the phosphorylation of STAT3 at tyrosine 705 (p-STAT3), AKT at serine 473 (p-AKT) and ERK1/2 at threonine 202 and tyrosine 204 (p-ERK). HMCL were stimulated alone with 10 ng/ml IL-6±200 ng/ml IGF-1 or stimulated in CC with HS5 stromal cells or primary BMSC with or without CYT387 treatment. For CC HS5 and primary BMSC were seeded into a 24 well plate at 2×105 cells/ml and allowed to establish for 4 hours, after which HMCL prestained with CD38 or CD138 FITC (BD) were added. MM cells were stimulated alone (10 ng/ml IL-6 or 5 ng/ml IL-6 and 100 ng/ml IGF-1), or in CC (direct CC with stroma or transwell (TW) CC with stroma) with or without either 60 minutes of CYT387 pretreatment or 15 minutes CYT387 co-treatment. After stimulation±treatment, MM cells were harvested and fixed with 2% paraformaldehyde for 10-30 minutes, washed then permeabilised with methanol overnight. Methanol was washed off and the cells were resuspended in p-STAT3 PE (BD), p-AKT PE (BD) or p-ERK (BD) and stained for 45-60 minutes at room temperature. Unbound antibody was washed off and the cells resuspended in 2% FBS PBS and acquired by FACS.

### Proliferation and Viability Assays

The viability and proliferation of CYT387 treated HMCL and untreated/vehicle controls were determined using various methods as described previously [26]. Proliferation was measured first using Celltiter 96 AQeous one solution cell proliferation assay MTS reagent (Promega) on a panel of 8 HMCL. Cells were cultured at 2.0×105 cell/ml in 100 μl fresh media in 96 well plates for 24, 48 and 72 hours with CYT387 (0.1-5 μM). 20 μl of MTS reagent was added for the final 4 hours of treatment and the plates were read at 490 nm using a Fluostar Optima plate reader (BMG Labtech). The viable cell numbers of a panel of 5 HMCL that were treated with CYT387 with and without IL-6 co-treatment was also measured by trypan blue staining and haemocytometer count. The HMCL NCI-H929, OCI-MY1 and U266 were then selected for further analysis. Apoptosis of CYT387 treated cells was assessed by FACS with Annexin-V and propidium iodide (PI) staining. HMCLs were treated for 24 or 72 hours with 1 or 5 μM CYT387 then harvested and washed in Annexin Buffer (0.01 HEPES, 0.14 M NaCl, 2.5 mM CaCl2, pH 7.4) and stained with Annexin-V FITC (Biosource) made up in Annexin Buffer for 30 minutes at room temperature. Unbound antibody was then washed off with Atmexin Buffer and cells were resuspended in Annexin Buffer with 62.5 ng/mi PI (Sigma-Aldrich) and analyzed by FACS.

For the synergy experiments HMCL were treated with CYT387 in combination with bortezomib or melphalan for 24 and 48 hours before being harvested and resuspended in FACS Buffer (0.5% HI FBS in PBS) supplemented with 62.5 ng/ml PI (Sigma-Aldrich). Cells were immediately analysed by FACS. The proportion of PI positive cells was quantitated by subtracting the background death of untreated cells. Single drug treated cells were compared to combination treated cells and synergism was calculated using Calcusyn software (Biosoft).

### Cell Cycling

The effect of CYT387 treatment on HMCL cell cycling was measured after 24 and 72 hours. CYT387 (1 or 5 μM) treated and untreated HMCL were harvested, washed in PBS and resuspended in 100 μl PBS. Cells were fixed with 1 ml of cold 70% ethanol while being vortexed. Tubes were stored at −20° C. until analysis. Once all samples were collected tubes were centrifuged at 500×g for 10 minutes, the supernatant was carefully removed and the cells washed in 5 ml PBS. After the final wash cells were resuspended 250-500 μl of PI/RNase staining buffer (BD) and incubated in the dark at room temperature for 15 minutes before being analyzed by FAGS.

### Data Analysis

All FACS data was acquired on a BD FACScalibur and data analysis done using Flowjo 7.6 Software (Treestar, USA). All statistical analysis was done using GraphPad Prism 5.03 software (USA).

### Results

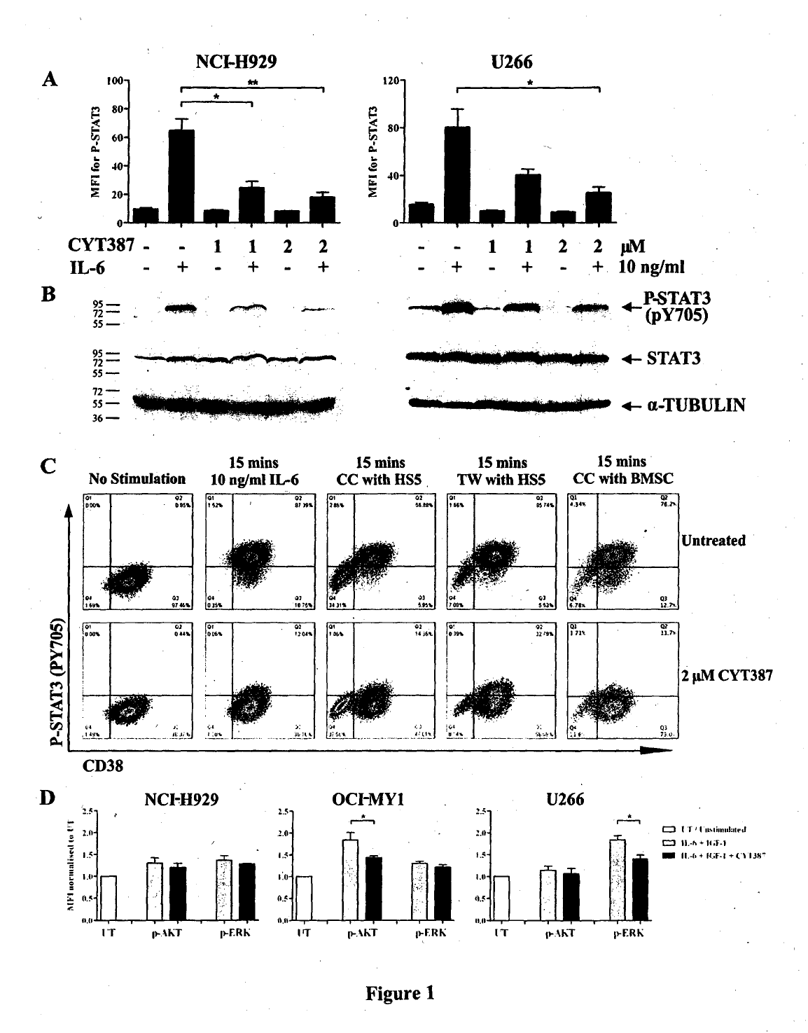

JAK/STAT signalling is inhibited by CYT3 87 IL-6 signalling through the JAK/STAT pathway is well characterized in MM cells with binding of IL-6 to its receptor inducing JAK2 to phosphorylate STAT3. The ability of CYT387 to inhibit JAK2 was first confirmed by measuring the level of STAT3 phosphorylation by western blotting and FACS. HMCL (NCI-H929, OCI-MY1 and U266) were incubated with CYT387 for 1 hour before being stimulated with IL-6 for 15 minutes to induce p-STAT3. CYT387 (0.5-2 μM) inhibited the phosphorylation of STAT3 in IL-6 stimulated samples as demonstrated by FACS (FIG. 1A) and confirmed by western blotting (FIG. 1B). Total STAT3 protein was unaffected.

Given the importance of the BMME in MM growth and survival it was important to establish if CYT387 could similarly modulate signalling in MM cells in CC with BMSC. This was done with both immortalized BMSC (HS5) and primary patient BMSC. In each case 15 minutes of CC with BMSC (with or without contact) was able to induce phosphorylation of STAT3 in the MM cells, whereas contemporaneous treatment with CYT387 dramatically reduced the amount of p-STAT3 in the HMCL (FIG. 1C). Thus, demonstrating that CYT387 is able to prevent STAT3 activation in MM cells induced by the soluble within the MM-BMSC microenvironment and also the contact mediated signalling provided to MM by BMSC.

### CYT387 Inhibits PI3K/AKT and Ras/MAPK Signalling

JAK signalling kinases are involved in many cellular pathways, which led us to investigate the effect of CYT387 on IL-6 and IGF-1 induced PI3K/AKT and Ras/MAPK signalling in NCI-H929, OCI-MY1 and U266 cells (FIG. 1D). OCI-MY1 showed distinct p-AKT activation after IL-6 and IGF-1 stimulation that was significantly reduced by CYT387 cotreatment. IL-6 and IGF-1 stimulation induced p-ERK in U266 cells which was significantly inhibited by CYT387. The levels of p-AKT and p-ERK showed only a small increase in response to IL-6 and IGF-1 stimulation in NCI-H929.

### CYT387 Inhibits Proliferation in HMCL

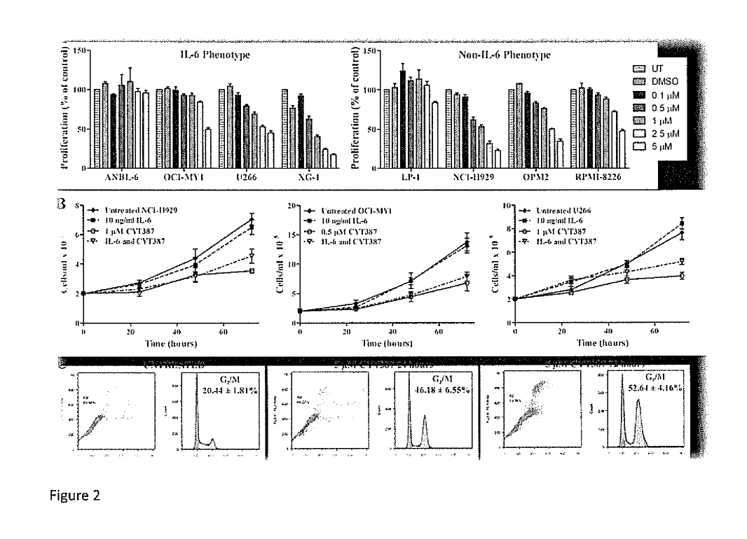

The effect of CYT387 (0.1-5 μM) on cell proliferation was measured by MTS assay at 24, 48 and 72 hours (FIG. 2A) on a panel of 8 HMCL (IL-6 non-responsive phenotype—LP-1, NCI-H929, OPM2, RPMI-8226 and IL-6 responsive phenotype—ANBL-6, OCI-MY1, U266 and XG-1). Six of 8 HMCL had a time and dose dependent response to CYT387 with inhibition in some HMCL within 24 hours. At 72 hours NCI-H929 and XG-1 were the most sensitive to CYT387 treatment.

Proliferation of HMCL cultured with CYT387 over 72 hours was also assessed by quantitation of viable cell number by haemocytometer (FIG. 2B). Because of the obvious relationship between IL-6 signalling and the inhibition of JAK2 by CYT387 the proliferation of 3 HMCL (NCI-H929, OCI-MY1 and U266) was measured with the addition of IL-6 and/or CYT387. The 3 HMCL proliferated well in complete media with or without supplementation with 10 ng/ml IL-6 and CYT387 (0.5-1 μM) was able to reduce the proliferation of HMCL in culture even in the presence of IL-6. CYT387 inhibited cell proliferation by 50% NCI-H929 (1 μM), 50% OCI-MY1 (0.5 μM) and 44% U266 (1 μM) after 72 hours.

Treatment of HMCL with CYT387 Results in an Accumulation of Cells in G2/M Phase of the Cell Cycle

The anti-proliferative effects of CYT387 were further characterised by evaluating the cell cycle of HMCL treated with CYT387. HMCL (NCI-H929, OCI-MY1 and U266) treated with CYT387 (1-5 μM) showed a marked accumulation of cells in the G2/M phase of the cell cycle. This was most pronounced in NCI-H929 cells (FIG. 2C), where there was more than a two fold increase in cells in G2/M in the drug treated sample compared to untreated or vehicle treated control after 24 and 72 hours treatment. An additional polyploid population was found in the CYT387 treated samples—suggesting that CYT387 treatment causes further aberration from the normal cell cycle of MM cells.

### CYT387 Induces Apoptosis in HMCL and Primary MM Cells

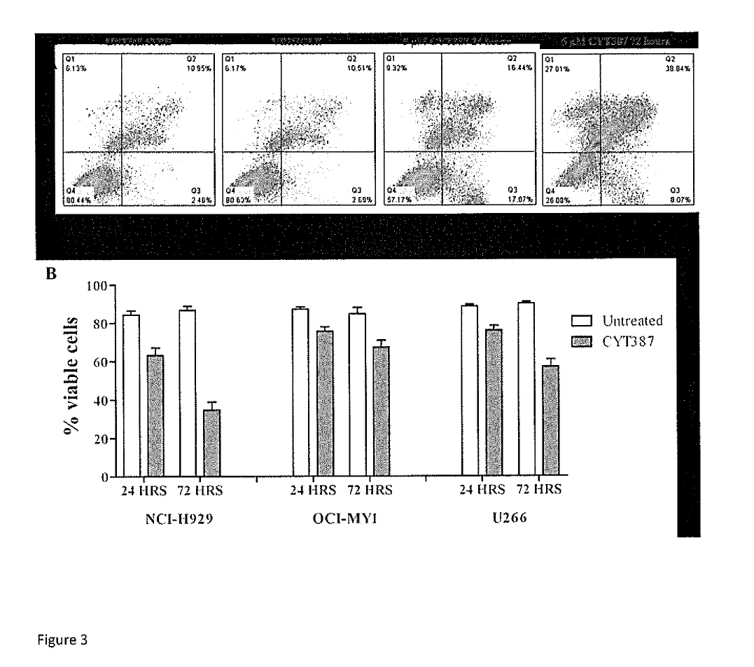

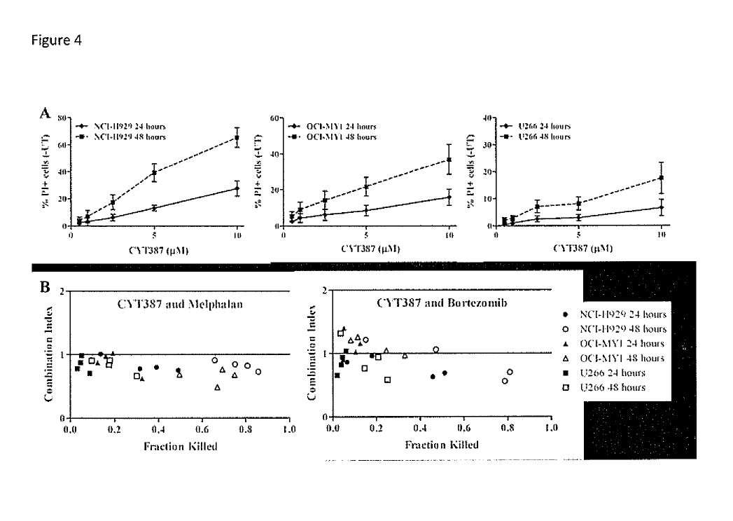

Apoptosis of CYT387 treated cells was investigated using Annexin-V/Propidium Iodide FACS staining in 3 HMCL (NCIH929, OCI-MY1 and U266). An increased proportion of apoptotic cells was detected in all 3 HMCL which was most evident in NCI-H929 (FIG. 3A), with a 21 and 52% reduction in viable cells detected at 24 and 72 hours, respectively, after treatment with 5 μM CYT387 (FIG. 3B). To assess CYT387 as part of a combination therapy NCI-H929, OCI-MY1 and U266 were treated with a range of doses of CYT387, bortezomib, melphalan to establish dose effect curves for each drug before combining with CYT387 and measuring the synergy using Calcusyn™ software. Dose effect curves generated for each compound (melphalan and bortezomib data not shown) show CYT387 (0.5-10 μM) induced apoptosis in 3 HMCL in a time and dose dependent manner (FIG. 4A). CYT387 displayed synergism with both bortezomib and melphalan but with variations in the level of synergy seen at differing drug dosages and analysis time-points (FIG. 4B).

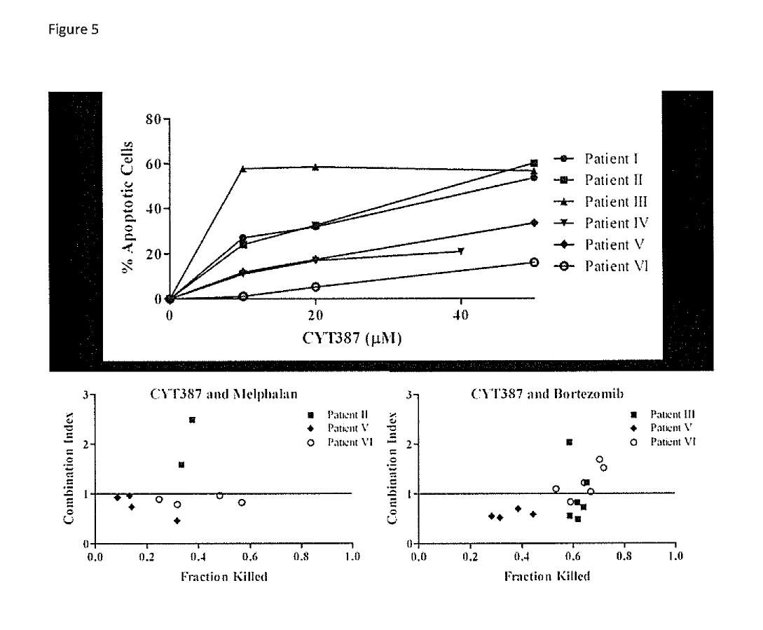

The effect of CYT387 on ex-vivo primary MM cells was also investigated, both as a single agent and as part of combination therapy. MM Patient BMA were cultured with various of doses of CYT387 (5-50 μM) for 24 and 48 hours after which the CD38+CD45− MM cell populations were assessed for apoptosis by flow cytometry. Six patients were treated and apoptosis was seen in between 5 and 59% of MM cells treated with 20 μM CYT387 after 48 hours (FIG. 5A). The effect of CYT387 in combination with melphalan and bortezomib was also investigated. CYT387 was seen to synergize with melphalan in ⅔ patients, and synergy was also observed with bortezomib in some patients/doses (FIG. 5B).

### Discussion

Multiple lines of evidence have confirmed the role of IL-6/JAK/STAT signalling in MM including experiments demonstrating the IL-6 dependence of some MM cells, the upregulation of proliferation of MM cells with IL-6, the inhibition of drug induced apoptosis by IL-6, and most important to this investigation, the direct induction of apoptosis in MM cells by inhibition of the IL-6/JAK/STAT pathway. These data and the abundance of IL-6 in the BMME makes JAK/STAT signalling a rational target for inhibition with new chemotherapeutics and is supported by preliminary in vitro studies that have demonstrated MM cell apoptosis induction via siRNA targeting of the JAK/STAT pathway [27]. Furthermore, the signal transduction role of JAKs in other important pathways [reviewed by 5] and the pro-survival effects of JAK mutations in other haematological malignancies [1-4] have already demonstrated the therapeutic potential of JAK inhibition. The therapeutic challenge is then inhibiting MM cells in the presence of IL-6 or BMSC. Here we have expanded upon previous work evaluating JAK inhibition in MM by studying a broader range of HMCL, by demonstrating that CYT387 can inhibit JAK-STAT activation in the context of coculture models and by demonstrating the impact of CYT387 on primary MM tumour cells. It has been hypothesized that in the earlier stages of MM that the malignant cells are predominantly CD45+, whereas in more advanced, drug-resistant disease, CD45− MM cells predominate [28]. Moreover, CD45− MM cells are considered less IL-6 responsive and express fewer IL-6 receptors than CD45+ MM [29]. Other studies of JAK inhibition have focused predominantly on CD45+ IL-6 responsive HMCL and while this is a logical focus for preliminary investigation it must be stressed that such target cell populations represents only a subset of MM cells. Furthermore, patients may demonstrate mixed populations of both CD45− and CD45+ MM cells [29]. Importantly, we have demonstrated the effectiveness of CYT387 against NCI-H929, a HMCL considered to have an IL-6 non-responsive phenotype suggesting that CYT387 will be effective against a range of MM phenotypes, whereas others [19] have reported only limited success against CD45− MM using alternative JAK inhibitors. Our data demonstrating synergy between CYT387 and bortezomib or melphalan against several HMCL and primary MM tumour cells confirms previously published work [19].

Available data suggests that the inhibition of JAK in MM cells may have downstream effects other than the direct inhibition of the JAK/STAT pathway. The importance of IL-6 in MM cell survival has been well characterized in terms of the JAK/STAT pathway but there is now increasing evidence of IL-6 induced PI3K/AKT activation [18, 30] and Ras/MAPK activation [20, 30-33] in various HMCL. Given the established importance of both the PI3K/AKT and Ras/MAPK pathways in addition to the JAK/STAT pathway in MM, small molecule inhibitors that could modulate all three could have enormous clinical potential.

Investigating the effects of JAK inhibition on the PI3K/AKT and Ras/MAPK pathways has yielded contrasting results. AZD1480 and AG490 have been shown to reduce IL-6 induced activation of Ras/MAPK [20, 22], but AG490 in the hands of others showed no decrease in IL-6 induced Ras/MAPK activation [31]. The JAK inhibitor INCB20 could inhibit IL-6 induced Ras/MAPK activation in MM.1S cells, but did not affect constitutive Ras/MAPK activation in INA-6 cells [18]. The inhibition of JAK with AG490 could also abrogate AKT activation in PTEN mutated OPM2 MM cells [30]. Similarly INCB20 could also inhibit IL-6 induced p-AKT, but had no effect on IGF-1 induced p-AKT in INA-6 cells [18]. Variability in the available data may be the result of differences in inhibitors and heterogeneity amongst HMCLs that are commonly studied. In our study there was a significant reduction in IL-6 and IGF-1 induced p-ERK in U266 cells, as well as a dramatic reduction of IL-6 and IGF-1 induced p-AKT in OCIMY1 cells supporting the data of others that suggests JAK inhibition may have broader anti-MM activity than would be initially expected. The inhibition of IL-6 induced JAK/STAT and PI3K/AKT signalling may also result in a reduction in IL-6R expression on the surface of MM cells [30], which could also lead to a reduction in the pro-survival effects of IL-6.

The profound anti-proliferative effect of CYT387 on various HMCL after a single dose is an important demonstration of its effectiveness. Furthermore, the ability of CYT387 to inhibit HMCL growth even in the presence of exogenous IL-6 which has been shown many times as a mediator of drug resistance [11-13] is noteworthy. This significant effect of JAK inhibition may be the result of HMCL having some dependence on JAK/STAT for proliferative signals, or more likely the involvement and subsequent inhibition of alterative signalling pathways mentioned above. The effect on proliferation is also seen in the cell cycling analysis which demonstrates that CYT387 can prevent cell cycling.

Also of interest was the additional polyploidy population (8n) induced by CYT387 treatment, which may suggest a role for JAK signalling leading to cell cycle regulation or may be the result of CYT387 inhibition of other kinases involved in cell cycle such as aurora B, aurora C, cyclin A or cyclin B as described by Pardanani et al., 2009. A direct or downstream effect of JAK inhibitors on cell cycling proteins is supported by studies on other JAK inhibitors; Scuto et al. found AZD1480 inhibited Cyclin D2 in two HMCL [22]. The induction of apoptosis in MM cells in primary marrow cocultures is a critical demonstration of the potential of CYT387. In contrast, other studies inhibiting IL-6 signalling have failed to demonstrate any convincing evidence of apoptosis when MM cells were treated in the presence of BMSC [32]. The investigators suggested that this might represent MM cell independence from IL-6 in the presence of BMSC. Our findings with CYT387 could be interpreted as either refuting the latter or alternatively being consistent with the capacity of JAK signalling inhibition to interrupt non-IL6 mediated survival pathways. Consistent with the latter was that the primary MM samples treated with CYT387 were representative of autologous whole marrow cocultures with the CD38+CD45− MM cells making up between 4 and 67% of treated cells. Therefore the demonstration that CYT387 was able to induce apoptosis of these heavily-treated MM cells was particularly encouraging.

### Additional Compound Evaluation

The compounds of formula Ib can tested in the mouse model of multiple myeloma as described in Dalton, W. and Anderson, K. C., Clinical Cancer Research, 2006:12(22), 6603-6610. Mouse models 5T2MM and 5T33MM are reported in Dalton et al as having clinical characteristics similar to the human disease, including localisation of multiple myeloma cells to the bone marrow, measurable M-protein in serum, induction of osteolytic bone disease, and increased angiogenesis in the marrow. Both models may therefore be used to test the compounds of formula Ib and allow for testing whether the compounds affect multiple myeloma cell homing before disease onset or after onset of the disease. Using these models, the effect of the compounds on the levels of serum M-protein may be determined and the effect of the compounds on inhibition of disease development such as reduction of osteolytic bone disease, reduction in tumor burden and improvement in survival, may also be determined.

In order to examine the effects of the compounds of formula Ib against a human myeloma, the compounds may be tested against xenograft models of human myeloma in mice. For these tests, the xenograft models of human myeloma tumors or cell lines may be transplanted into, for example, SCID-Hu or NOD/SCID mice. The SCID-Hu model may be used to study myeloma in a human microenvironment and the impact of the compounds of formula Ib on the reproducible growth of primary myeloma cells may also be studied. The NOD/SCID model involves labelling multiple myeloma cells with green fluorescent protein. This model may be used to follow disease progression.

### REFERENCES

1. Kralovics, R., F. Passamonti, A. S. Buser, et al., A gain-of-function mutation of JAK2 in myeloproliferative disorders. N Engl J Med, 2005. 352(17): p. 1779-90.
2. Levine, R. L., M. Wadleigh, J. Cools, et al., Activating mutation in the tyrosine kinase JAK2 in polycythemia vera, essential thrombocythemia, and myeloid metaplasia with myelofibrosis. Cancer Cell, 2005. 7(4): p. 387-97.
3. Scott, L. M., W. Tong, R. L. Levine, et al., JAK2 exon 12 mutations in polycythemia vera and idiopathic erythrocytosis. N Engl J Med, 2007. 356(5): p. 459-68.
4. Mullighan, C. G., J. Zhang, R. C. Harvey, et al., JAK mutations in high-risk childhood acute lymphoblastic leukemia. Proc Natl Acad Sci USA, 2009. 106(23): p. 9414-8.
5. Rane, S. G. and E. P. Reddy, Janus kinases: components of multiple signaling pathways. Oncogene, 2000. 19(49): p. 5662-79.
6. French, J. D., D. K. Walters, and D. F. Jelinek, Transactivation of gp130 in myeloma cells. J Immunol, 2003. 170(7): p. 3717-23.
7. Berger, L. C., T. S. Hawley, J. A. Lust, S. J. Goldman, and R. G. Hawley, Tyrosine phosphorylation of JAK-TYK kinases in malignant plasma cell lines growth-stimulated by interleukins 6 and 11. Biochem Biophys Res Commun, 1994. 202(1): p. 596-605.
8. Gomez-Benito, M., P. Balsas, X. Carvajal-Vergara, et al., Mechanism of apoptosis induced by IFN-alpha in human myeloma cells: role of Jak1 and Bim and potentiation by rapamycin. Cell Signal, 2007. 19(4): p. 844-54.
9. Jelinek, D. F., G. J. Ahmann, P. R. Greipp, et al., Coexistence of aneuploid subclones within a myeloma cell line that exhibits clonal immunoglobulin gene rearrangement: clinical implications. Cancer Res, 1993. 53(21): p.5320-7.
10. Zhang, X. G., J. P. Gaillard, N. Robillard, et al., Reproducible obtaining of human myeloma cell lines as a model for tumor stem cell study in human multiple myeloma. Blood, 1994. 83(12): p. 3654-63.
11. Cheung, W. C. and B. Van Ness, The bone marrow stromal microenvironment influences myeloma therapeutic response in vitro. Leukemia, 2001. 15(2): p. 264-71.
12. Moreaux, J., E. Legouffe, E. Jourdan, et al., BAFF and APRIL protect myeloma cells from apoptosis induced by interleukin 6 deprivation and dexamethasone. Blood, 2004. 103(8): p. 3148-57.
13. Perez, L. E., N. Parquet, K. Shain, et al., Bone marrow stroma confers resistance to Apo2 ligand/TRAIL in multiple myeloma in part by regulating c-FLIP. J Immunol, 2008. 180(3): p. 1545-55.
14. Klein, B., X. G. Zhang, M. Jourdan, et al., Paracrine rather than autocrine regulation of myeloma-cell growth and differentiation by interleukin-6. Blood, 1989. 73(2): p. 517-26.
15. Tyner, J. W., T. G. Bumm, J. Deininger, et al., CYT387, a novel JAK2 inhibitor, induces hematologic responses and normalizes inflammatory cytokines in murine myeloproliferative neoplasms. Blood, 2010. 115(25): p. 5232-5240.
16. Pardanani, A., T. Lasho, G. Smith, C. J. Burns, E. Fantino, and A. Tefferi, CYT387, a selective JAK1/JAK2 inhibitor: in vitro assessment of kinase selectivity and preclinical studies using cell lines and primary cells from polycythemia vera patients. Leukemia, 2009. 23(8): p. 1441-5.
17. Burns, C. J., D. G. Bourke, L. Andrau, et al., Phenylaminopyrimidines as inhibitors of Janus kinases (JAKs). Bioorg Med Chem Lett, 2009. 19(20): p. 5887-92.
18. Burger, R., S. Le Gouill, Y. T. Tai, et al., Janus kinase inhibitor INCB20 has antiproliferative and apoptotic effects on human myeloma cells in vitro and in vivo. Mol Cancer Ther, 2009. 8(1): p. 26-35.
19. Li, J., M. Favata, J. A. Kelley, et al., INCB16562, a JAK1/2 selective inhibitor, is efficacious against multiple myeloma cells and reverses the protective effects of cytokine and stromal cell support. Neoplasia, 2010. 12(1): p.28-38.
20. De Vos, J., M. Jourdan, K. Tarte, C. Jasmin, and B. Klein, JAK2 tyrosine kinase inhibitor tyrphostin AG490 downregulates the mitogen-activated protein kinase (MAPK) and signal transducer and activator of transcription (STAT) pathways and induces apoptosis in myeloma cells. Br J Haematol, 2000. 109(4): p. 823-8.
21. Alas, S. and B. Bonavida, Inhibition of constitutive STAT3 activity sensitizes resistant non-Hodgkin's lymphoma and multiple myeloma to chemotherapeutic drug-mediated apoptosis. Clin Cancer Res, 2003. 9(1): p. 316-26.
22. Scuto, A., P. Krejci, L. Popplewell, et al., The novel JAK inhibitor AZD1480 blocks STAT3 and FGFR3 signaling, resulting in suppression of human myeloma cell growth and survival. Leukemia, 2010.
23. Pedranzini, L., T. Dechow, M. Berishaj, et al., Pyridone 6, a pan-Janus-activated kinase inhibitor, induces growth inhibition of multiple myeloma cells. Cancer Res, 2006. 66(19): p. 9714-21.
24. Ferrajoli, A., S. Faderl, Q. Van, et al., WP1066 disrupts Janus kinase-2 and induces caspase-dependent apoptosis in acute myelogenous leukemia cells. Cancer Res, 2007. 67(23): p. 11291-9.
25. Khong, T., J. Sharkey, and A. Spencer, The effect of azacitidine on interleukin-6 signaling and nuclear factorkappaB activation and its in vitro and in vivo activity against multiple myeloma. Haematologica, 2008. 93(6): p.860-9.
26. Monaghan, K., T. Khong, G. Smith, and A. Spencer, CYT997 Causes apoptosis in human multiple myeloma. Invest New Drugs, 2009.
27. Chatterjee, M., T. Stuhmer, P. Herrmann, K. Bommert, B. Dorken, and R. C. Bargou, Combined disruption of both the MEKERK and the IL-6R/STAT3 pathways is required to induce apoptosis of multiple myeloma cells in the presence of bone marrow stromal cells. Blood, 2004. 104(12): p. 3712-21.
28. Kumar, S., S. V. Rajkumar, T. Kimlinger, P. R. Greipp, and T. E. Witzig, CD45 expression by bone marrow plasma cells in multiple myeloma: clinical and biological correlations. Leukemia, 2005. 19(8): p. 1466-70.
29. Hata, H., H. Xiao, M. T. Petrucci, J. Woodliff, R. Chang, and J. Epstein, Interleukin-6 gene expression in multiple myeloma: a characteristic of immature tumor cells. Blood, 1993. 81(12): p. 3357-64.
30. Thabard, W., M. Collette, M. P. Mellerin, et al., IL-6 upregulates its own receptor on some human myeloma cell lines. Cytokine, 2001. 14(6): p. 352-6.
31. Puthier, D., R. Bataille, and M. Amiot, IL-6 up-regulates mcl-1 in human myeloma cells through JAK/STAT rather than ras/MAP kinase pathway. Eur J Immunol, 1999. 29(12): p. 3945-50.
32. Chatterjee, M., D. Honemann, S. Lentzsch, et al., In the presence of bone marrow stromal cells human multiple myeloma cells become independent of the IL-6/gp130/STAT3 pathway. Blood, 2002. 100(9): p. 3311-8.
33. Ferlin, M., N. Noraz, C. Hertogh, J. Brochier, N. Taylor, and B. Klein, Insulin-like growth factor induces the survival and proliferation of myeloma cells through an interleukin-6-independent transduction pathway. Br J Haematol, 2000. 111(2): p. 626-34.

1. A method of treating a subject presenting with multiple myeloma at a stage characterized by an increase in the prevalence of MM cells that are IL-6 non-responsive and/or have a CD45− phenotype, comprising administering to the subject an amount of a compound of formula Ib: 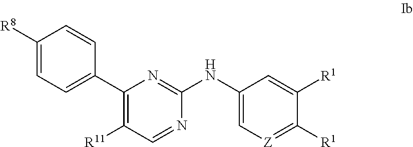
wherein
Z is independently selected from N and CH;
R¹ is independently selected from H, halogen, OH, CONHR², CON(R²)₂, CF₃, R²OR², CN, morpholino, thiomorpholinyl, thiomorpholino-1,1-dioxide, substituted or unsubstituted piperidinyl, substituted or unsubstituted piperazinyl, imidazolyl, substituted or unsubstituted pyrrolidinyl and C₁₋₄alkylene wherein the carbon atoms are optionally replaced with NR^{Y} and/or O substituted with morpholino, thiomorpholinyl, thiomorpholino-1,1-dioxide, substituted or unsubstituted piperidinyl, substituted or unsubstituted piperazinyl, imidazolyl or substituted or unsubstituted pyrrolidinyl;
R² is substituted or unsubstituted C₁₋₄alkyl;
R^{Y} is H or substituted or unsubstituted C₁₋₄alkyl;
R⁸ is R^{X}CN;
R^{X} is substituted or unsubstituted C₁₋₄alkylene wherein up to 2 carbon atoms can be optionally replaced with CO, NSO₂R¹, NR^{Y}, CONR^{Y}, SO, SO₂ or O;
R¹¹ is H or C₁₋₄alkyl, or an enantiomer thereof or a pharmaceutically acceptable salt thereof effective to reduce the viability of said MM cells.

2. A method of treating a subject presenting with multiple myeloma, comprising the steps of (1) selecting, for treatment, a subject diagnosed with an increase in the prevalence of MM cells that are IL-6 non-responsive, and/or have a CD45− phenotype; and (2) administering to the subject an amount of the compound of formula Ib defined in claim 1, or a pharmaceutically acceptable salt thereof effective to reduce the viability of said MM cells.

3. A method for treating a subject presenting with multiple myeloma, comprising the steps of (1) obtaining a sample of MM cells from said subject; (2) analyzing said MM cells for the prevalence of cells that are IL-6 non-responsive and/or have a CD45− phenotype; (3) selecting for treatment a subject in which the prevalence of said MM cells is increased relative to subjects at an early stage of multiple myeloma, and (4) administering to the selected subject an amount of the compound of formula Ib defined in claim 1, or a pharmaceutically acceptable salt thereof effective to reduce the viability of said MM cells.

4. The method according to claim 1, wherein the compound of formula Ib has the structure shown below: 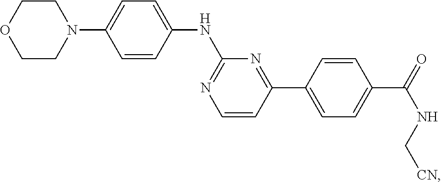 [SMILES: N#CCNC(=O)c1ccc(-c2ccnc(Nc3ccc(N4CCOCC4)cc3)n2)cc1]
or a pharmaceutically acceptable salt thereof.

5. The method according to claim 4, wherein the subject is afflicted with newly diagnosed or relapsed multiple myeloma.

6. The method according to claim 5, wherein the subject presents with a population of MM cells in which at least 10% of said cells are CD45−.

7. The method according to claim 6, further comprising administering to the subject a second therapeutic agent selected from melphalan and bortezomib.

8. An article of manufacture, comprising a container comprising the compound of formula Ib defined in claim 1, or a pharmaceutically acceptable salt thereof.

9. A kit comprising the compound of formula Ib defined in claim 1, or a pharmaceutically acceptable salt thereof.

10. The method according to claim 2, wherein the compound of formula Ib has the structure shown below: 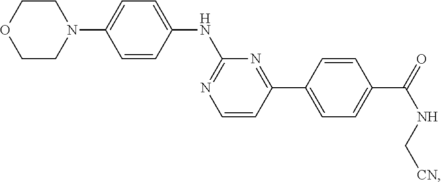 [SMILES: N#CCNC(=O)c1ccc(-c2ccnc(Nc3ccc(N4CCOCC4)cc3)n2)cc1]
or a pharmaceutically acceptable salt thereof.

11. The method according to claim 10, wherein the subject is afflicted with newly diagnosed or relapsed multiple myeloma.

12. The method according to claims 11, wherein the subject presents with a population of MM cells in which at least 10% of said cells are CD45−.

13. The method according to claim 12, further comprising administering to the subject a second therapeutic agent selected from melphalan and bortezomib.

14. The method according to claim 3, wherein the compound of formula Ib has the structure shown below: 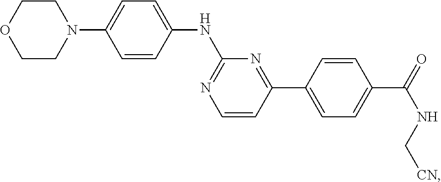 [SMILES: N#CCNC(=O)c1ccc(-c2ccnc(Nc3ccc(N4CCOCC4)cc3)n2)cc1]
or a pharmaceutically acceptable salt thereof.

15. The method according to claim 14, wherein the subject is afflicted with newly diagnosed or relapsed multiple myeloma.

16. The method according to claims 15, wherein the subject presents with a population of MM cells in which at least 10% of said cells are CD45−.

17. The method according to claim 16, further comprising administering to the subject a second therapeutic agent selected from melphalan and bortezomib.

18. The article of manufacture according to claim 8, wherein the compound of formula Ib has the structure shown below: 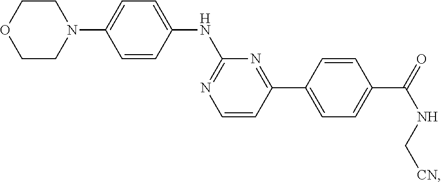 [SMILES: N#CCNC(=O)c1ccc(-c2ccnc(Nc3ccc(N4CCOCC4)cc3)n2)cc1]
or a pharmaceutically acceptable salt thereof.

19. The kit according to claim 9, wherein the compound of formula Ib has the structure shown below: 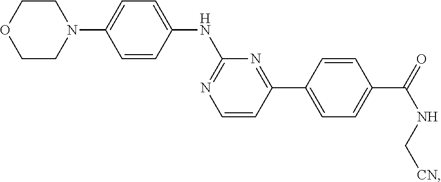 [SMILES: N#CCNC(=O)c1ccc(-c2ccnc(Nc3ccc(N4CCOCC4)cc3)n2)cc1]
or a pharmaceutically acceptable salt thereof.
